(19) United States
(12) Patent Application Publication
(10) Pub. No.: US 2022/0016409 A1
(43) Pub. Date: Jan. 20, 2022

(54) STERILIZABLE PERITONEAL DIALYSIS CONNECTION DEVICE

(71) Applicant: The Johns Hopkins University, Baltimore, MD (US)

(72) Inventors: Sarah LEE, Baltimore, MD (US); James QIN, Baltimore, MD (US); Anna Claire BAILEY, Baltimore, MD (US); Eugene OH, Brooklyn, NY (US); Tejasvi DESAI, Pleasanton, CA (US); Giang HOANG, Baltimore, MD (US); Dylan HIRSCH, Rockville, MD (US); Hallie HORVATH, Washington, DC (US)

(73) Assignee: The Johns Hopkins University, Baltimore, MD (US)

(21) Appl. No.: 17/309,273

(22) PCT Filed: Oct. 29, 2019
(86) PCT No.: PCT/US2019/058568

§ 371 Date: May 13, 2021

Related U.S. Application Data
(60) Provisional application No. 62/767,250, filed on Nov. 14, 2018.

(51) Int. Cl. A61M 39/18; A61M 1/28; A61M 39/12

(52) U.S. Cl. CPC A61M 39/18; A61M 39/12; A61M 1/285

(57) ABSTRACT

A sterilizable connector may include a sterilization fluid reservoir to provide a sterilization fluid. The sterilizable connector may include a connector. The connector may include a first end to receive a first tube to connect to a medical device (e.g., directly or via one or more intervening components). The connector may include a second end to receive a second tube to connect to a patient (e.g., directly or via one or more intervening components). The connector may include a body to couple the first end to the second end to enable fluid transfer between the first tube and the second tube. The connector may include a body opening to couple to the sterilization fluid reservoir. The sterilizable connector may include a sterilization fluid injector to inject the sterilization fluid from the sterilization fluid reservoir to the connector.

### RELATED APPLICATION(S)

This application claims priority to U.S. Provisional Patent Application No. 62/767,250, filed on Nov. 14, 2018, and entitled “STERILIZABLE PERITONEAL DIALYSIS CONNECTION DEVICE,” the content of which is incorporated by reference herein in its entirety.

### BACKGROUND

Different types of dialysis techniques may be used as a treatment for end-stage renal disease (ESRD). For example, in hemodialysis (HD), a patient may be treated at a treatment center, such as a hospital or a dialysis clinic. In this case, the patient may attend a treatment session at the treatment center three or more times each week. During a treatment session, a hemodialysis machine may remove a patient's blood from the patient's body using a first needle at a first injection site, may filter the patient's blood, and may provide the patient's blood back into the patient's body using a second needle at a second injection site. In this way, the hemodialysis machine may perform external filtration of the patient's blood. However, attendance at multiple treatment sessions each week may be inconvenient, stressful, painful, and/or cost-prohibitive for a patient.

Another type of dialysis technique is peritoneal dialysis (PD). In peritoneal dialysis, a patient may be surgically implanted with a catheter in the patient's peritoneal cavity. In this case, the patient may connect the surgically implanted catheter to an external tube, referred to as a transfer set, and may connect the transfer set to a disposable cassette tube connected to a peritoneal dialysis cycler, which may inject dialysate fluid and remove waste fluid to perform dialysis. Using peritoneal dialysis may enable the patient to perform dialysis at home, thereby improving patient outcomes and satisfaction and reducing healthcare costs and inconvenience.

### BRIEF DESCRIPTION OF THE DRAWINGS

FIGS. 1A-1D are diagrams of an overview of an example implementation of a sterilizable connector described herein.

FIG. 2 is a flow chart of an example process for sterilization for peritoneal dialysis.

FIGS. 3A-3E, 4-7, and 8A-8D are diagrams of example implementations of a sterilizable connection described herein.

### DETAILED DESCRIPTION

The following detailed description of example implementations refers to the accompanying drawings. The same reference numbers in different drawings may identify the same or similar elements.

As described above, peritoneal dialysis may enable a patient to perform at-home dialysis, which may result in reduced costs, increased convenience, improved patient outcomes, and improved patient satisfaction relative to hemodialysis, which may be performed at a treatment center. However, peritoneal dialysis may be associated with a high risk of infection, such as of a peritoneal membrane of the patient, which is a condition referred to as peritonitis. This may result from a patient or caregiver, who is not trained in sterilization techniques or who fails to comply with training in sterilization techniques, connecting tubing of a peritoneal dialysis system in a non-sterile environment. For example, although a transfer set and cassette tubing may be sterilized during manufacture, when connecting a transfer set to cassette tubing of the peritoneal dialysis system, a patient may touch ends of the transfer set and the cassette tubing with bare hands. This may result in bacteria or other microbes being disposed onto tubing surfaces. During peritoneal dialysis, dialysate may be exposed to the bacteria or other microbes during injection into a patient, thereby exposing the patient to a risk of infection.

Some implementations described herein provide a sterilizable connector to enable sterilization for peritoneal dialysis. For example, a sterilizable connector may include a body with a first end to connect to a transfer set and a second end to connect to cassette tubing. Further, the sterilizable connector may include a sterilization fluid reservoir connected to the body and a sterilization fluid injector to inject the sterilization fluid into the body, the transfer set, and the cassette tubing. In this way, the sterilization fluid may sterilize surfaces of the sterilizable connector, the transfer set, and the cassette tubing, thereby reducing a likelihood of patient infection relative to directly connecting the transfer set and the cassette tubing, as described above. Although some implementations described herein are described in terms of connection to a transfer set and cassette tubing in a peritoneal dialysis system, the sterilizable connector may be used for other connections associated with a peritoneal dialysis system, for other connections in other contexts outside of a peritoneal dialysis system, and/or the like. Although some implementations described herein are described in terms of use in a peritoneal dialysis system, implementations described herein may be used in another context for which sterilized connectors are used, such as home intravenous anti-infective therapies (HIVAT), at-home chemotherapy, and/or the like.

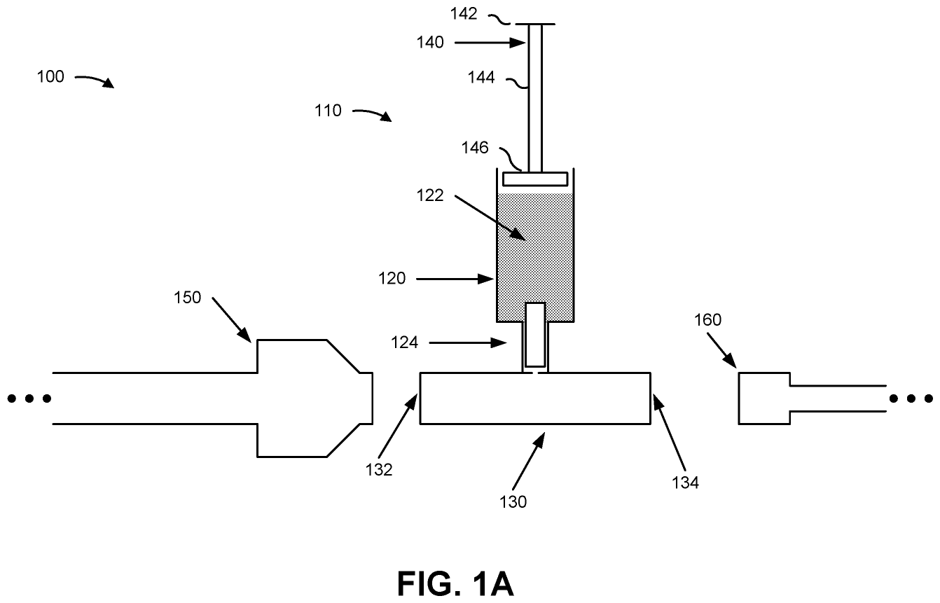

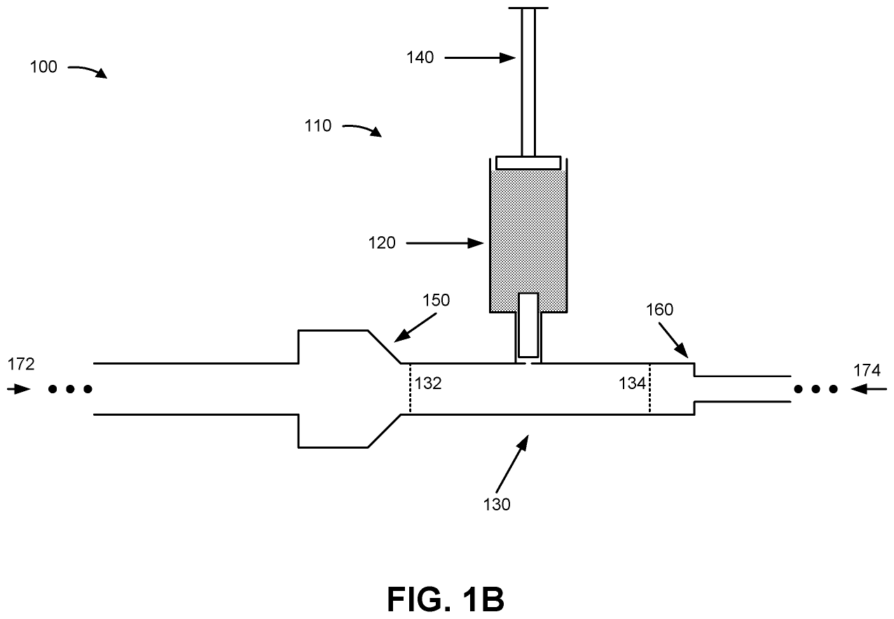

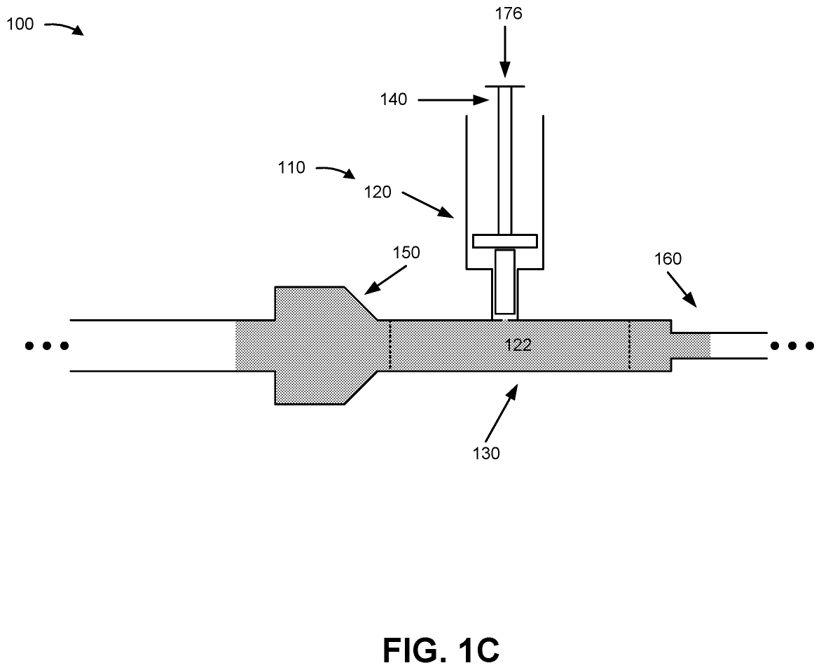

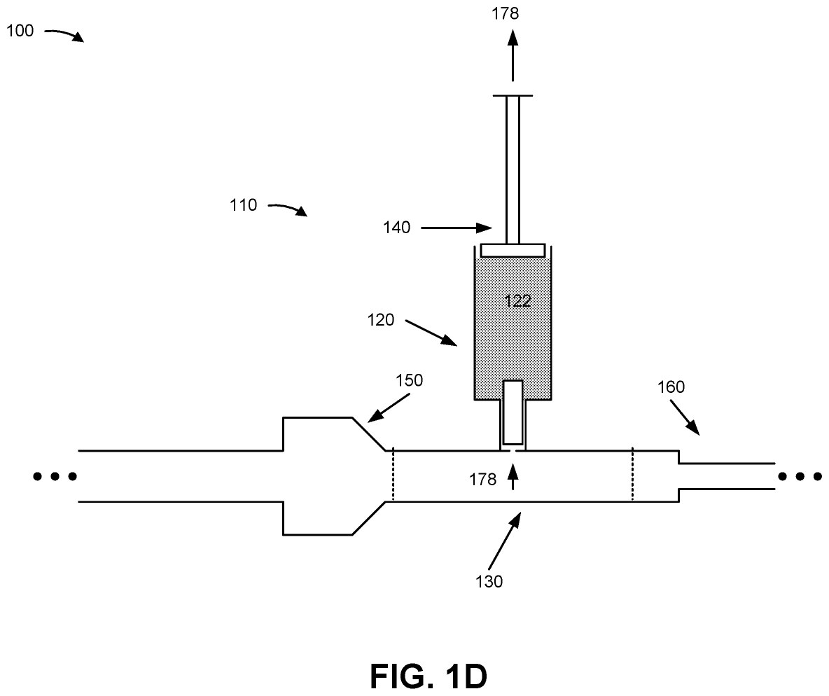

FIGS. 1A-1D are diagrams of an overview of an example implementation 100 described herein. As shown in FIG. 1A, example 100 may include a sterilizable connector 110, a cassette tube 150 (e.g., which may be connected to a peritoneal dialysis cycler), and a transfer set 160 (e.g., which may be connected to a patient). Although some implementations described herein are described in terms of a sterilizable connector 110 being positioned at a connection between a cassette tube 150 and a transfer set 160, other positions are contemplated, such as at a connection between transfer set 160 and a dialysate bag tube connected to a dialysate bag. Other connections for which sterilization may be advantageous also are contemplated.

As further shown in FIG. 1A, sterilizable connector 110 may include a sterilization fluid reservoir 120, a connector 130, and a sterilization fluid injector 140. In some implementations, sterilization fluid reservoir 120 may be a barrel of a syringe or other injector. In some implementations, sterilization fluid reservoir 120 may include a sterilization fluid 122, that may be injected into connector 130. For example, sterilization fluid reservoir 120 may include an anti-microbial fluid to sterilize surfaces of connector 130, cassette tube 150, transfer set 160, and/or the like, thereby reducing a risk of patient infection. In some implementations, sterilization fluid reservoir 120 may be sized to hold a particular amount of sterilization fluid 122, such as 1 milliliter (ml), 5 ml, 10 ml, 20 ml, and/or the like. In some implementations, sterilization fluid reservoir 120 may include a valve 124. For example, valve 124 may enable injection of sterilization fluid 122, removal of sterilization fluid 122, and/or the like.

In some implementations, valve 124 may enable disconnection of sterilization fluid reservoir 120 and sterilization fluid injector 140. For example, after injection of sterilization fluid 122, as described in more detail below, sterilization fluid reservoir 120 and sterilization fluid injector 140 may be detached from connector 130, and valve 124 may seal an opening of connector 130. In this way, by detaching a portion of sterilizable connector 110, a likelihood of sterilizable connector 110 interfering with tubes of the peritoneal dialysis system and/or causing patient discomfort is reduced. Moreover, sterilization fluid reservoir 120 may be replaceable by attaching new sterilization fluid reservoirs 120 each time a patient is to perform peritoneal dialysis, thereby reducing an inconvenience and/or difficulty of use associated with connecting connector 130.

In some implementations, sterilization fluid reservoir 120 may include a restrictor piece or dwell mechanism. For example, sterilization fluid reservoir 120 may include a rubber slider, an angled flap, a latched lever, and/or the like to control a depth to which sterilization fluid injector 140 may be inserted into sterilization fluid reservoir 120, a height to which sterilization fluid injector 140 may be removed from sterilization fluid reservoir 120, a dwell time for sterilization fluid 122 to remain in connector 130, a rate at which sterilization fluid injector 140 injects and/or removes sterilization fluid 122, and/or the like. In some implementations, sterilizable connector 110 may include an indicator to indicate when the dwell time is elapsed, such as an indicator device. Additionally, or alternatively, a configured chemical reaction may be used to indicate when the dwell time is elapsed.

In some implementations, connector 130 may be a body disposed between cassette tube 150 and transfer set 160. For example, a first end 132 of connector 130 may receive and connect to cassette tube 150 and a second end 134 of connector 130 may receive and connect to transfer set 160, as described in more detail herein. In some implementations, connector 130 may form a tube connecting cassette tube 150 and transfer set 160.

In some implementations, connector 130 may include a body opening to receive sterilization fluid 122. In some implementations, connector 130 may include one or more other openings. For example, connector 130 may include another opening to receive one or more wash reservoirs that includes a wash fluid. In this case, the wash fluid may be injected into connector 130 after injecting the sterilization fluid, and the wash fluid may displace the sterilization fluid, thereby removing sterilization fluid 122 from connector 130, from cassette tube 150, from transfer set 160, and/or the like. In this way, a likelihood of sterilization fluid 122 being inserted into a patient is reduced.

In some implementations, sterilization fluid injector 140 may include a plunger flange 142, a plunger 144, and a plunger seal 146. For example, sterilization fluid injector 140 may form a plunger that may be inserted into and removed from sterilization fluid reservoir 120 to inject sterilization fluid 122 into connector 130 and remove sterilization fluid 122 from connector 130. In some implementations, sterilization fluid injector 140 may include a dampener, which may restrict a rate at which sterilization fluid 122 is injected into connector 130, a rate at which sterilization fluid 122 is removed from connector 130, and/or the like. In some implementations, the dampener may be a spring to automatically raise sterilization fluid injector 140 to remove sterilization fluid 122 from connector 130. In some implementations, sterilization fluid injector 140 may include a particular type of button mechanism that may form at least a portion of plunger flange 142. For example, sterilization fluid injector 140 may include a pull button, a push button, a squeeze button, a twist mechanism, and/or the like. In some implementations, the button mechanism may be a reusable mechanism and at least a portion of sterilization fluid injector 140 may be a disposable mechanism (e.g., connector 130, sterilization fluid reservoir 120, and/or the like may be disposable to maintain sterilization).

As shown in FIG. 1B, and by reference number 172, first end 132 of sterilizable connector 110 may receive cassette tube 150. For example, a patient may attach an end of cassette tube 150 to first end 132, and may seal a connection. In this case, first end 132 and/or cassette tube 150 may include an attachment portion, such as a cap, a clamp, a screw end, and/or the like to seal the connection. As shown by reference number 174, second end 134 of sterilizable connector 110 may receive transfer set 160. For example, a patient may attach an end of transfer set 160 to second end 134, and may seal a connection. In this case, second end 134 and/or transfer set 160 may include an attachment portion, such as a cap, a clamp, a screw end, and/or the like to seal the connection. In some implementations, sterilizable connector 110 may be connected in another context. For example, sterilizable connector 110 may receive, at first end 132, a first tube (e.g., associated with a medical device or patient) and, at second end 134, a second tube (e.g., associated with a medical device or patient) to enable sterilization of a connection between the first tube and the second tube. In some implementations, sterilizable connector 110 may be connected to the medical device or patient via one or more other intervening components, such as one or more valves, one or more other connectors, one or more other tubes, and/or the like. For example, although the first tube is described as connecting to, for example, the medical device, the first tube may connect directly to the medical device, indirectly to the medical device (e.g., via one or more intervening components), and/or the like.

As shown in FIG. 1C, and by reference number 176, sterilization fluid injector 140 may inject sterilization fluid 122 into connector 130, cassette tube 150, transfer set 160, and/or the like. For example, a patient may depress a plunger of sterilization fluid injector 140 to inject sterilization fluid 122. In this case, sterilization fluid 122 may remain in, for example, connector 130, cassette tube 150, transfer set 160, and/or the like for a threshold period of time to enable sterilization of surfaces of connector 130, cassette tube 150, transfer set 160, and/or the like. In this way, sterilizable connector 110 reduces a likelihood of patient infection by enabling sterilization of a connection between cassette tube 150 and transfer set 160.

As shown in FIG. 1D, and by reference number 178, sterilization fluid injector 140 may remove sterilization fluid 122 from connector 130, cassette tube 150, transfer set 160, and/or the like. For example, a patient may raise a plunger of sterilization fluid injector 140 to remove sterilization fluid 122. In this case, the patient may detach sterilization fluid reservoir 120, sterilization fluid injector 140, and/or the like, as described above. In some implementations, a sterilization fluid retractor may be separate from sterilization fluid injector 140. For example, another plunger may retract sterilization fluid 122 from connector 130. Additionally, or alternatively, a wash fluid reservoir and a wash fluid injector may inject wash fluid into connector 130 to displace sterilization fluid 122, thereby retracting sterilization fluid 122. Additionally, or alternatively, a peritoneal dialysis cycler attached to cassette tube 150 may retract sterilization fluid 122. Additionally, or alternatively, as shown, sterilization fluid injector 140 may be the sterilization fluid retractor.

In some implementations, based on retracting sterilization fluid 122, peritoneal dialysis may be performed. For example, based on sealing valve 124, dialysate fluid may be injected into a patient by a peritoneal dialysis cycler via cassette tube 150, connector 130, transfer set 160, a surgically implanted catheter connected to transfer set 160, and/or the like. Similarly, the peritoneal dialysis cycler may cause waste fluid to be removed from a patient via cassette tube 150, connector 130, transfer set 160, and/or the like. In this way, peritoneal dialysis may be performed using sterilizable connector 110 with a reduced risk of patient infection.

As indicated above, FIGS. 1A-1D are provided merely as an example. Other examples may differ from what is described with regard to FIGS. 1A-1D.

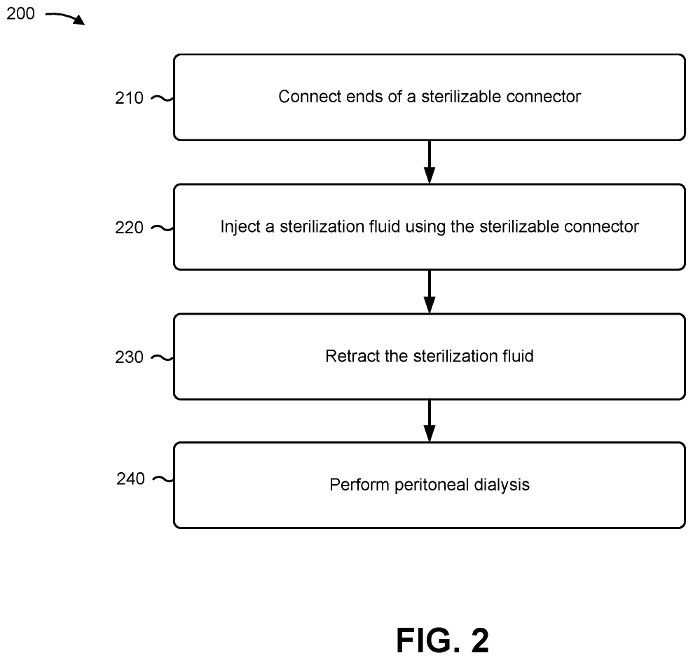

FIG. 2 is a flow chart of an example process 200 for sterilization for peritoneal dialysis. In some implementations, one or more process blocks of FIG. 2 may be performed by a patient or caregiver using a sterilizable connector.

As shown in FIG. 2, process 200 may include connecting ends of a sterilizable connector (block 210). For example, the patient may connect the ends of the sterilizable connector, as described above. In some implementations, the patient may attach a cassette tube of a peritoneal dialysis cycler to a first opening of a body portion of a sterilizable connector. In some implementations, the patient may attach a transfer set to a second opening of the body portion of the sterilizable connector. In some implementations, ends of the sterilizable connector may be connected to, for example, the cassette tube and the transfer set without patient interaction. For example, a medical robotics device may automatically connect the cassette tube and the transfer set to the sterilizable connector. In some implementations, an end of the sterilizable connector may be connected to a dialysate fluid bag via a tube.

As further shown in FIG. 2, process 200 may include injecting a sterilization fluid using the sterilizable connector (block 220). For example, the patient may use the sterilizable connector to inject the sterilization fluid, as described above. In some implementations, the patient may inject the sterilization fluid into a third opening of a body portion of the sterilizable connector, such that the sterilization fluid is in contact with at least a portion of the cassette tube, at least a portion of the transfer set, and at least a portion of the body portion. In some implementations, a medical robotics device may automatically inject the sterilization fluid into the body portion of the sterilizable connector. In some implementations, the sterilizable connector may be positioned between and/or used to sterilize one or more other interconnection points of adjacent components in a fluid transfer path, such as the fluid transfer path described herein, another fluid transfer path, and/or the like.

As further shown in FIG. 2, process 200 may include retracting the sterilization fluid (block 230). For example, the patient may use the sterilizable connector to retract the sterilization fluid, as described above. In some implementations, the patient may use the sterilizable connector to retract the sterilization fluid, to displace or dilute the sterilization fluid with a wash fluid, and/or the like. In some implementations, the patient may close a closeable valve of the sterilizable connector to seal the third opening of the body portion of the sterilizable connector and to enable detachment of a portion of the sterilizable connector.

As further shown in FIG. 2, process 200 may include performing peritoneal dialysis (block 240). For example, the patient may perform peritoneal dialysis, as described above. In some implementations, the patient may use a peritoneal dialysis cycler of a peritoneal dialysis cycler system (e.g., that includes the peritoneal dialysis cycler, one or more tubes, and/or the like) to perform peritoneal dialysis via a fluid transfer path that includes the cassette tube connected to a peritoneal dialysis cassette, the body portion of the sterilizable connector, the transfer set, and/or the like. Additionally, or alternatively, the fluid transfer path may include a dialysate fluid bag tube connected to a dialysate fluid bag.

Process 200 may include additional implementations, such as any single implementation or any combination of implementations described above and/or in connection with one or more other processes described elsewhere herein.

Although FIG. 2 shows example blocks of process 200, in some implementations, process 200 may include additional blocks, fewer blocks, different blocks, or differently arranged blocks than those depicted in FIG. 2. Additionally, or alternatively, two or more of the blocks of process 200 may be performed in parallel.

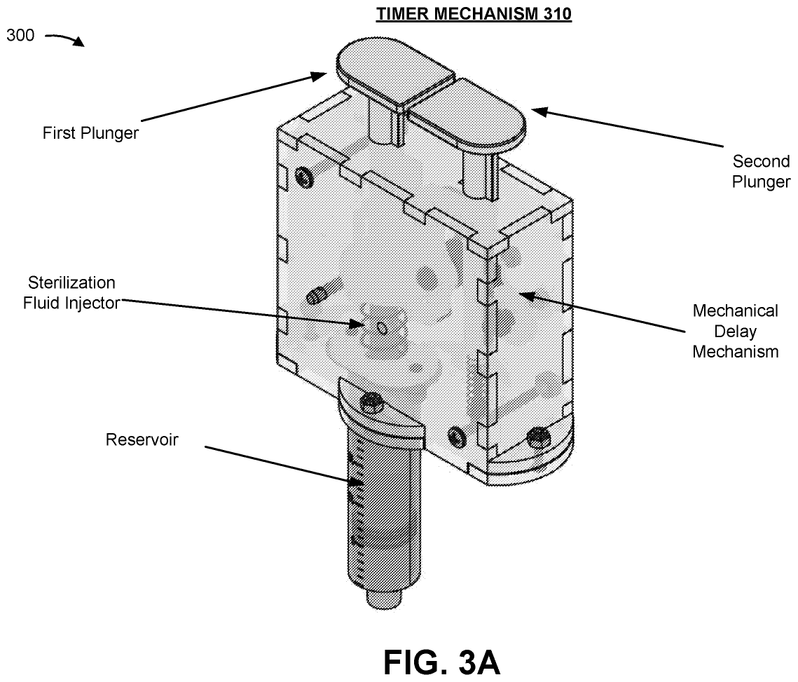

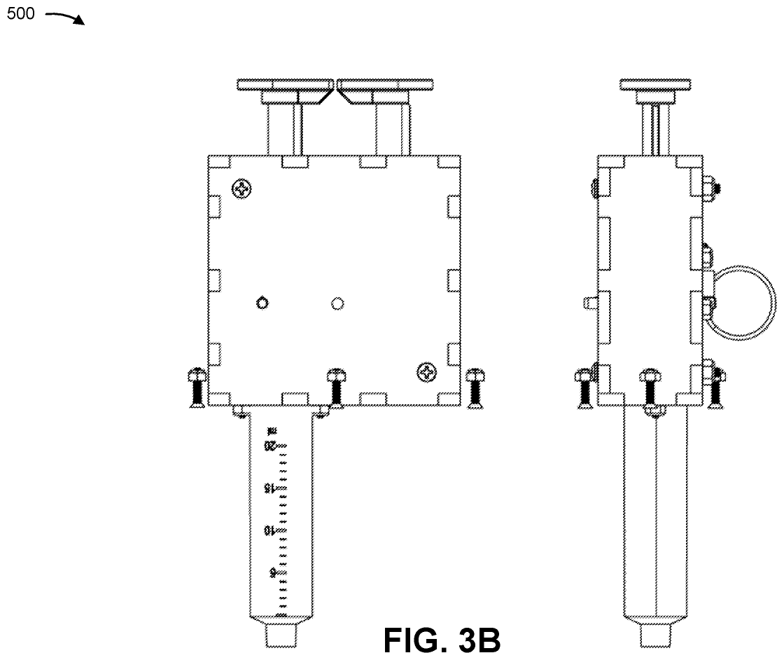

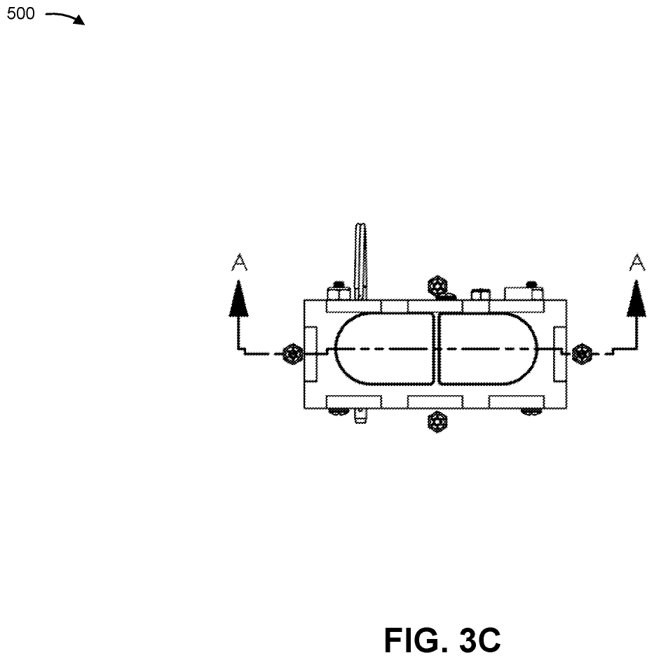

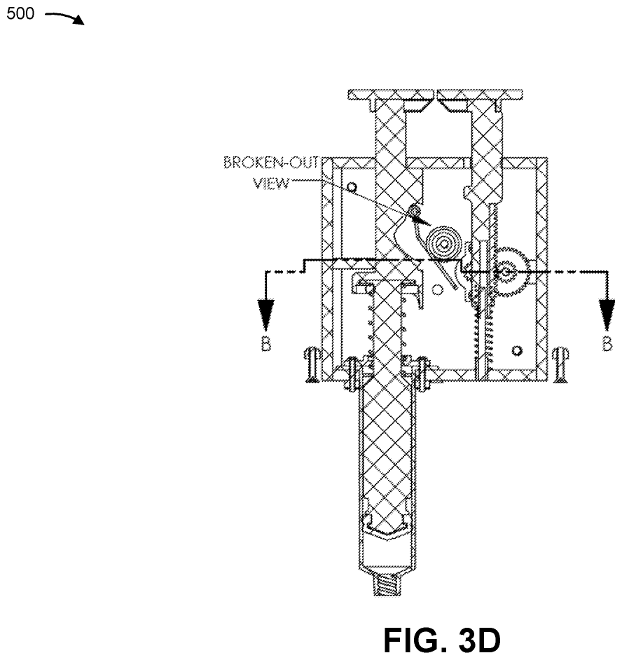

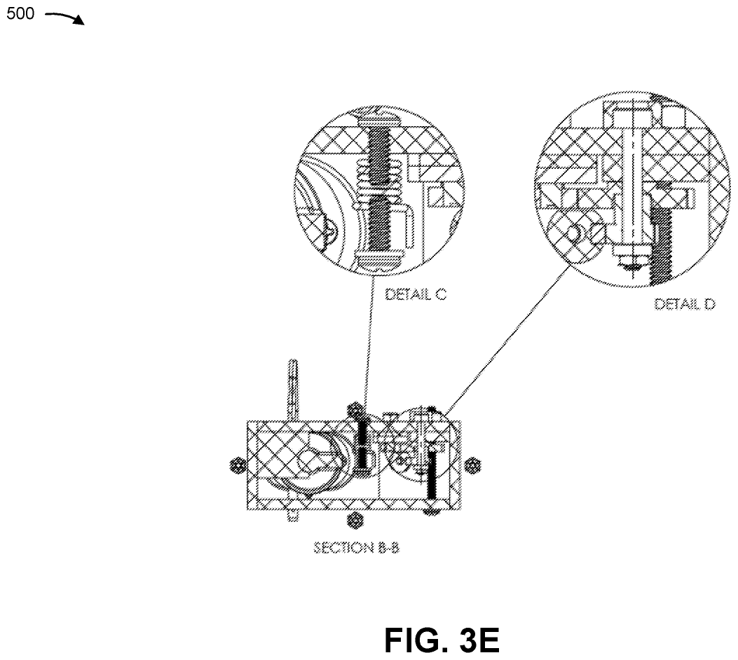

FIGS. 3A-3E are diagrams of an example implementation 300 described herein.

As shown in FIGS. 3A-3E, example implementation 300 may include a timer mechanism 310 to enable automated control of injection and/or removal of sterilization fluid. In this case, a user may depress a first plunger to inject a sterilization fluid into a sterilizable connector, and a second plunger, which may attach to a mechanical delay mechanism. The mechanical delay mechanism of the second plunger may hold the first plunger in a depressed position, using a pivot arm hooked into an indentation in the first plunger, for a set amount of time. In some implementations, the set amount of time (e.g., a configured dwell time) may be configured using a spring and a set of rotating gears that interface with a rack to allow the pivot arm to be unhooked. After expiration of the set amount of time, the mechanical delay mechanism may release the second plunger by unhooking the pivot arm from the indentation in the first plunger. In this case, a spring mechanism of the first plunger may raise the first plunger, thereby removing the sterilization fluid from the connector. Although some implementations are described in terms of a mechanical delay mechanism, other delay mechanisms may be used, such as an electrical delay mechanism, an electro-mechanical delay mechanism, a micro-electro-mechanical system (MEMS) delay mechanism, and/or the like.

As indicated above, FIGS. 3A-3E are provided merely as an example. Other examples may differ from what is described with regard to FIGS. 3A-3E.

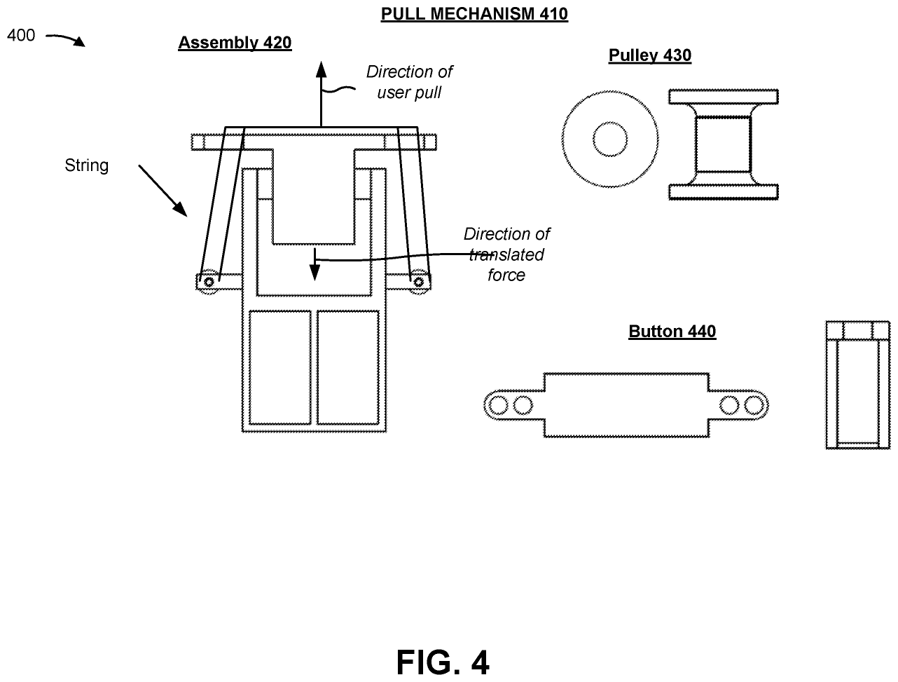

FIG. 4 is a diagram of an example implementation 400 described herein.

As shown in FIG. 4, a pull mechanism 410 may be provided to enable injection and/or removal of a sterilization fluid. Pull mechanism 410 may include an assembly 420, a pulley 430, and a button 440. In some implementations, pull mechanism 410 may correspond to and/or attach to a sterilization fluid injector. Pull mechanism 410 translates a user pull on, for example, a string (e.g., that interfaces with pulley 430 and button 440) to apply force on a plunger of assembly 420, thereby enabling injection of a sterilization fluid using a sterilizable connector. In some implementations, pull mechanism 410 may include a string, a wire, or another type of connector.

As indicated above, FIG. 4 is provided merely as an example. Other examples may differ from what is described with regard to FIG. 4.

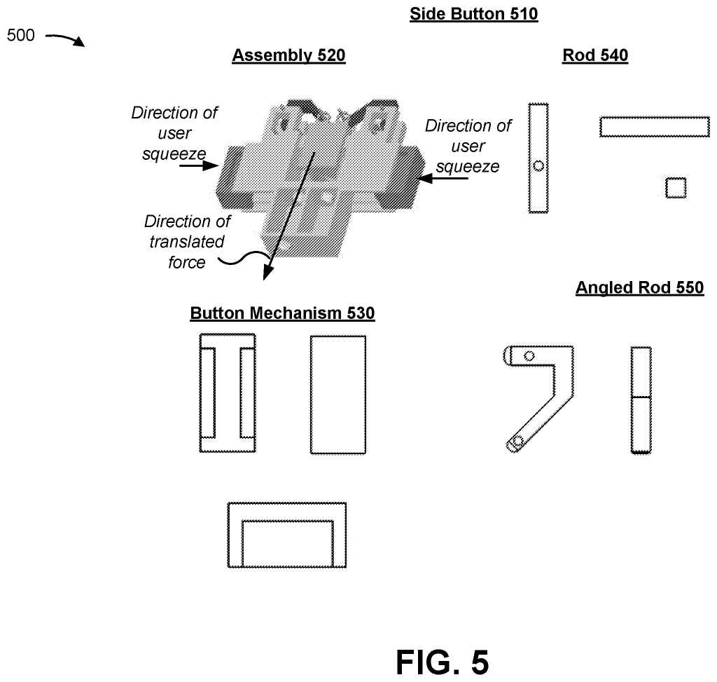

FIG. 5 is a diagram of an example implementation 500 described herein.

As shown in FIG. 5, example implementation 500 includes a side button 510. Side button 510 may include an assembly 520, which includes a button mechanism 530, a rod 540, and angled rod 550. Side button 510 translates a user squeeze action to force on a plunger of assembly 520 to enable injection of a sterilization fluid using a sterilizable connector.

As indicated above, FIG. 5 is provided merely as an example. Other examples may differ from what is described with regard to FIG. 5.

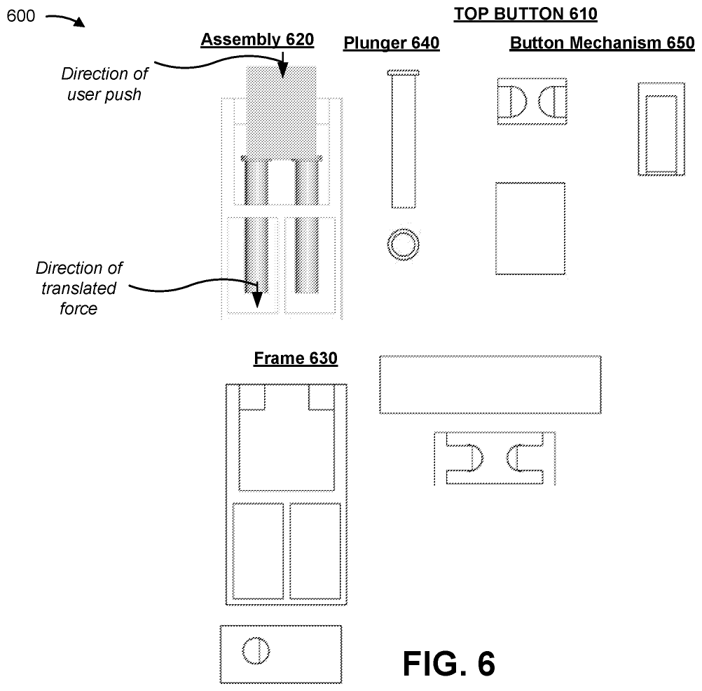

FIG. 6 is a diagram of an example implementation 600 described herein.

As shown in FIG. 6, example implementation 600 includes a top button 610. Top button 610 includes an assembly 620, a frame 630, a plunger 640, and a button mechanism 650. Top button 610 translates a user push action to force on a plunger of assembly 620 to enable injection of a sterilization fluid using a sterilizable connector.

As indicated above, FIG. 6 is provided merely as an example. Other examples may differ from what is described with regard to FIG. 6.

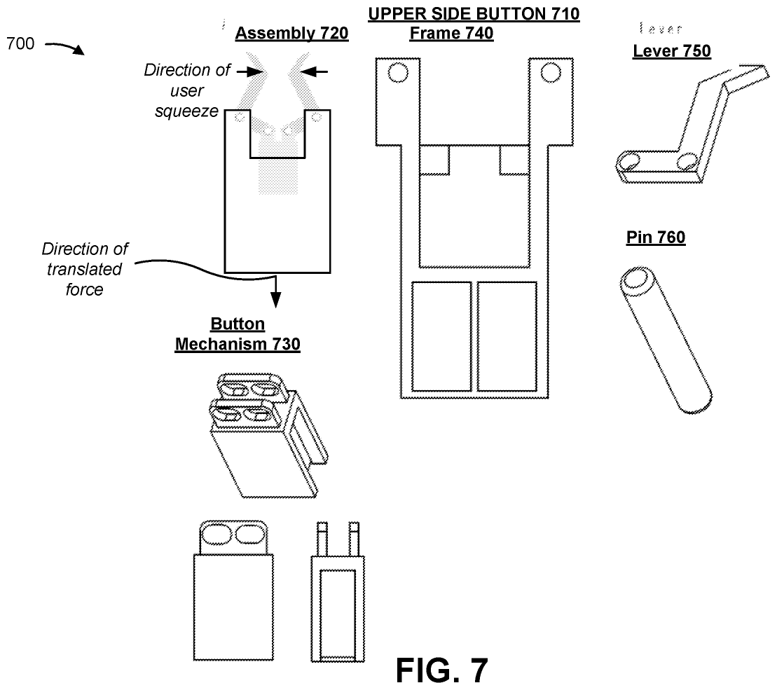

FIG. 7 is a diagram of an example implementation 700 described herein.

As shown in FIG. 7, example implementation 700 includes an upper side button 710. Upper side button 710 includes an assembly 720, a button mechanism 730, a frame 740, a lever 750, and a pin 760. Upper side button 710 translates a user squeeze action to force on a plunger of assembly 720 to enable injection of a sterilization fluid using a sterilizable connector.

As indicated above, FIG. 3 is provided merely as an example. Other examples may differ from what is described with regard to FIG. 7.

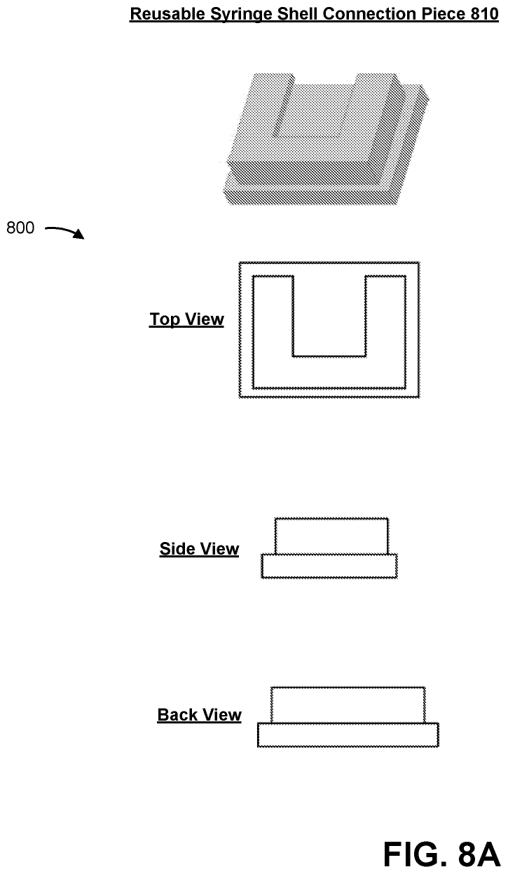

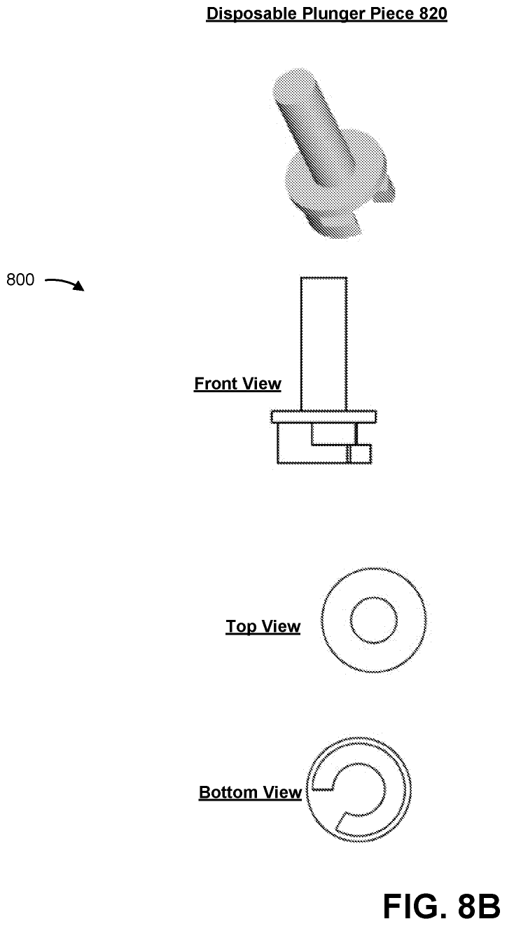

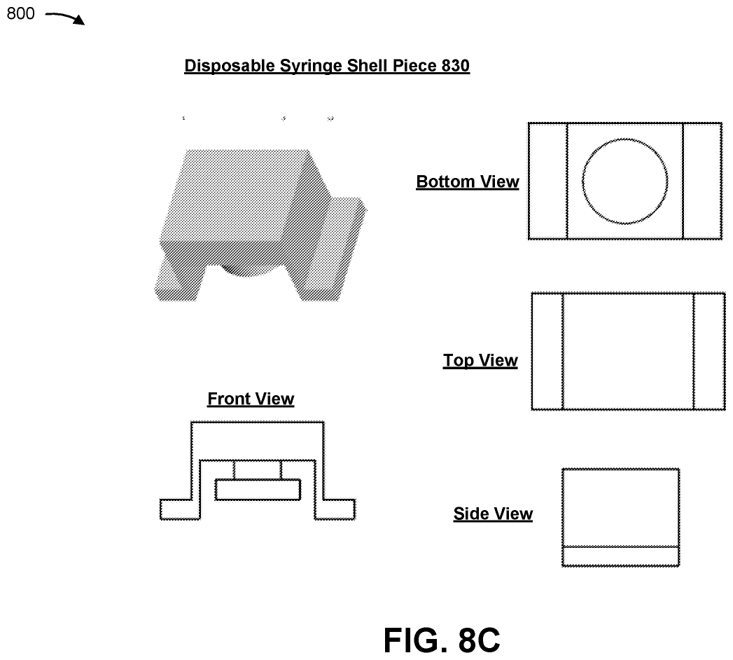

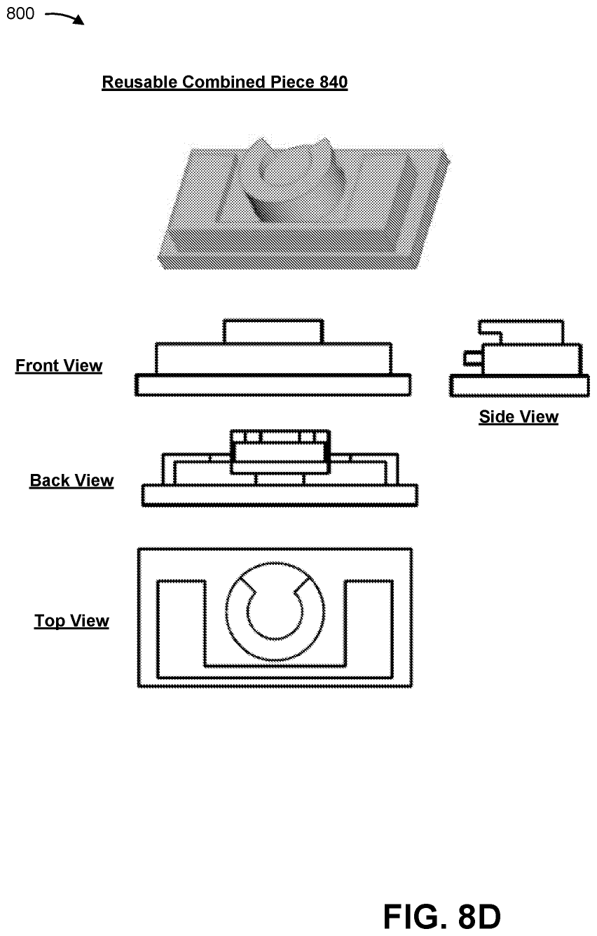

FIGS. 8A-8D are diagrams of an example implementation 800 described herein.

As shown in FIGS. 8A-8D, example implementation 800 includes a reusable syringe shell connection piece 810, a disposable plunger piece 820, a disposable syringe shell piece 830, and a reusable combined piece 840. As shown in FIGS. 8A-8D, example implementation 800 includes multiple detachable parts, thereby enabling disposable, sterilized components to be used for a sterilizable connection. For example, syringe shell connection piece 810 may be a reusable component onto which a disposable plunger piece 820 is attached, a disposable syringe shell piece 830 is attached, and/or the like.

As indicated above, FIGS. 8A-8D are provided merely as an example. Other examples may differ from what is described with regard to FIGS. 8A-8D.

The foregoing disclosure provides illustration and description, but is not intended to be exhaustive or to limit the implementations to the precise form disclosed. Modifications and variations may be made in light of the above disclosure or may be acquired from practice of the implementations.

As used herein, satisfying a threshold may, depending on the context, refer to a value being greater than the threshold, more than the threshold, higher than the threshold, greater than or equal to the threshold, less than the threshold, fewer than the threshold, lower than the threshold, less than or equal to the threshold, equal to the threshold, or the like.

Even though particular combinations of features are recited in the claims and/or disclosed in the specification, these combinations are not intended to limit the disclosure of various implementations. In fact, many of these features may be combined in ways not specifically recited in the claims and/or disclosed in the specification. Although each dependent claim listed below may directly depend on only one claim, the disclosure of various implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise.

What is claimed is:

1. A sterilizable connector, comprising:
a sterilization fluid reservoir to provide a sterilization fluid;
a connector, comprising:
a first end to receive a first tube,
a second end to receive a second tube,
a body to couple the first end to the second end to enable fluid transfer between the first tube and the second tube,
a body opening to couple to the sterilization fluid reservoir; and
a sterilization fluid injector to inject the sterilization fluid from the sterilization fluid reservoir to the connector.

2. The sterilizable connector of claim 1, wherein at least one of the first tube or the second tube is to connect to at least one of:
a peritoneal dialysis cassette,
a dialysate fluid bag,
a transfer set.

3. The sterilizable connector of claim 1, wherein at least one of the first tube or the second tube is at least one of:
a transfer set,
a cassette tubing, or
a dialysate bag tubing.

4. The sterilizable connector of claim 1, wherein the sterilization fluid injector is to inject the sterilization fluid to sterilize at least a portion of at least one of: the connector, the first tube, or the second tube.

5. The sterilizable connector of claim 1, further comprising:
a sterilization fluid retractor to retract the sterilization fluid from the connector.

6. The sterilizable connector of claim 5, wherein the sterilization fluid injector is the sterilization fluid retractor.

7. The sterilizable connector of claim 1, further comprising:
a closeable valve coupled to the body opening to enable detachment of the sterilization fluid injector from the connector.

8. The sterilizable connector of claim 1, wherein the body opening is a first body opening, and
wherein the connector comprises:
at least one second body opening to receive a wash fluid to displace the sterilization fluid in the connector.

9. The sterilizable connector of claim 8, further comprising:
a wash fluid reservoir to couple to the second body opening; and
a wash fluid injector to inject the wash fluid into the second body opening to displace the sterilization fluid in the connector.

10. The sterilizable connector of claim 1, further comprising:
a dampener to dampen an injection of or a removal of the sterilization fluid.

11. The sterilizable connector of claim 1, further comprising:
a dwell mechanism to cause a configured dwell time of the sterilization fluid in the connector.

12. The sterilizable connector of claim 11, further comprising:
an indicator to indicate that the configured dwell time has elapsed.

13. A method comprising:
attaching a first tube of a peritoneal dialysis cycler system to a first opening of a body portion of a sterilizable connector;
attaching a second tube to a second opening of the body portion of the sterilizable connector; and
injecting, using the sterilizable connector, a sterilization fluid into a third opening of the body portion of the sterilizable connector, such that the sterilization fluid is in contact with at least a portion of the body portion, at least a portion of the first tube, and at least a portion of the second tube.

14. The method of claim 13, wherein at least one of the first tube or the second tube is at least one of:
a cassette tube,
a dialysate bag tube, or
a transfer set tube.

15. The method of claim 13, further comprising:
retracting the sterilization fluid from the sterilizable connector; and
closing a valve of the sterilizable connector to seal the third opening.

16. The method of claim 13, further comprising:
injecting a wash fluid into the body portion to displace or dilute the sterilization fluid.

17. The method of claim 13, further comprising:
performing peritoneal dialysis,
wherein a fluid transfer path for the peritoneal dialysis includes the body portion.

18. A sterilizable connector, comprising:
a sterilization fluid reservoir to provide a sterilization fluid;
a connector attached to the sterilization fluid reservoir; and
a sterilization fluid injector to inject the sterilization fluid from the sterilization fluid reservoir to the connector,
wherein the sterilization fluid injector includes a button mechanism.

19. The sterilizable connector of claim 18, wherein the button mechanism includes a mechanical delay mechanism.

20. The sterilizable connector of claim 18, wherein the button mechanism is at least one of: a push button, a pull button, a squeeze button, or a twist mechanism.
